(19) United States
(12) Patent Application Publication
(10) Pub. No.: US 2019/0059814 A1
(43) Pub. Date: Feb. 28, 2019

(54) Electroencephalogram Measurement System Using Virtual Reality Device and Band

(71) Applicants: Gyoung Mo Kim, Seoul (KR); Joon Hyun Jeon, Gyeonggi-do (KR)

(72) Inventors: Gyoung Mo Kim, Seoul (KR); Joon Hyun Jeon, Gyeonggi-do (KR)

(21) Appl. No.: 16/046,561

(22) Filed: Jul. 26, 2018

(30) Foreign Application Priority Data
Aug. 24, 2017 (KR) 10-2017-0107406

(51) Int. Cl. A61B 5/00; A61B 5/0484; G06F 3/01

(52) U.S. Cl. CPC A61B 5/6803; G06F 3/015; A61B 5/0484

(57) ABSTRACT

An electroencephalogram measurement system using a Virtual Reality (VR) device and a band includes an electroencephalogram measurement apparatus comprising a cover that is worn on a user's head, and a protruding sensor protruding from the cover at a designated location; a band that is put on the electroencephalogram measurement apparatus, the band having a sensor hole formed in correspondence to a location at which the protruding sensor protrudes; and a VR device coupled to a front portion of the band, and disposed around the user's eyes, wherein, after the electroencephalogram measurement apparatus is put on the user's head, the band and the VR device are worn on the user's head while the protruding sensor of the electroencephalogram measurement apparatus passes through the sensor hole of the band.

### CROSS-REFERENCE TO RELATED APPLICATION

This application claims the benefit of Korean Patent Application No. 10-2017-0107406, filed on Aug. 24, 2017, in the Korean Intellectual Property Office, the disclosure of which is incorporated herein in its entirety by reference.

### BACKGROUND

### 1. Field

One or more embodiments relate to an electroencephalogram measurement system using a Virtual Reality (VR) device and a band, and more particularly, to an electroencephalogram measurement system using a VR device and a band, which is capable of preventing interference between the band coupled to the VR device and an electroencephalogram measurement apparatus through sensor holes formed in the band in order to use the band in the electroencephalogram measurement apparatus.

### 2. Description of the Related Art

With active studies of cognitive science and development of technologies, various techniques for measuring and analyzing the brain in the social science field, such as functional magnetic resonance imaging (fMRI), functional near infrared spectroscopy (fNIRS), electroencephalography (EEG), and eye-tracking, have been developed. The techniques for measuring and analyzing the brain are performed by applying a stimulus to a subject according to an experiment or an experimental design and then measuring the brain waves of the subject through an electroencephalogram measurement apparatus.

The electroencephalogram measurement apparatus, which is worn on the subject's head, includes sensors protruding above the subject's head. The sensors of the electroencephalogram measurement apparatus are disposed at specific locations where brain waves emerge from the subject's brain to measure the brain waves, and connected to connecting lines to transmit the measured brain waves to an analysis apparatus.

In order to measure brain waves, it is necessary to apply a stimulus to a subject according to an experiment or an experimental design, and in order to apply such a stimulus, a Virtual Reality (VR) device is used. The VR device provides virtual reality to the subject to make the subject sense reality in a virtual environment through various factors (high-definition images, head tracking, high quality of sound, etc.) for raising the sense of reality in the virtual environment.

However, when the VR device is used in the electroencephalogram measurement apparatus, the following problems may be generated.

The VR device, which is disposed around a user's eyes, has a very heavy weight to implement virtual reality. In order to measure brain waves, the electroencephalogram measurement apparatus is worn on a subject's head, and the VR device is mounted on the electroencephalogram measurement apparatus. At this time, the VR device is mounted on the electroencephalogram measurement apparatus by a fixing band, etc.

More specifically, the fixing band of the VR device is placed on the electroencephalogram measurement apparatus having sensors protruding above the subject's head. When the fixing band of the VR device is placed on the electroencephalogram measurement apparatus, the fixing band of the VR device is located on some sensors of the electroencephalogram measurement apparatus. Accordingly, the sensors located below the fixing band of the VR device are pressed by the heavy weight of the VR device, and thus, a pressure difference between the sensors and the other sensors on which the fixing band is not located is made. The pressure difference between the sensors causes measurement errors or inadequate data values.

Also, connecting lines that are connected to the sensors of the electroencephalogram measurement apparatus interfere with the fixing band of the VR device, and accordingly, there are difficulties in connecting the connecting lines to the sensors. Furthermore, the pressure of the fixing band due to the weight of the VR device has an adverse effect on the connecting lines.

### SUMMARY

One or more embodiments include an electroencephalogram measurement system using a virtual reality (VR) device and a band, which is capable of preventing interference between the band coupled to the VR device and an electroencephalogram measurement apparatus through sensor holes formed in the band in order to use the band in the electroencephalogram measurement apparatus.

Additional aspects will be set forth in part in the description which follows and, in part, will be apparent from the description, or may be learned by practice of the presented embodiments.

According to one or more embodiments, an electroencephalogram measurement system for measuring brain waves using a VR device includes: an electroencephalogram measurement apparatus including a cover that is worn on a user's head, and a protruding sensor protruding from the cover at a designated location; a band that is put on the electroencephalogram measurement apparatus, the band having a sensor hole formed in correspondence to a location at which the protruding sensor protrudes; and a VR device coupled to a front portion of the band, and disposed around the user's eyes, wherein, after the electroencephalogram measurement apparatus is put on the user's head, the band and the VR device are worn on the user's head while the protruding sensor of the electroencephalogram measurement apparatus passes through the sensor hole of the band.

The band may include an upward band diverging into a plurality of pieces to extend toward the top of the user's head from the VR device, and a horizontal band diverging into two pieces to extend in a horizontal direction from the VR device, wherein the upper band is put on the user's head earlier than the horizontal band. The electroencephalogram measurement system may further include a hook on which the upward band and the horizontal band converge, wherein the horizontal band is detachably coupled to the hook.

The sensor hole may include a first sensor hole penetrating an inner portion of the band, and a second sensor hole penetrating an edge portion of the band. A mold may be coupled to the sensor hole along an inner circumference surface of the sensor hole.

The electroencephalogram measurement system may further include a connecting line connected to the protruding sensor and configured to transmit a signal generated by the protruding sensor, wherein the connection line is connected to the protruding sensor after the band and the VR device are worn on the user's head.

### BRIEF DESCRIPTION OF THE DRAWINGS

These and/or other aspects will become apparent and more readily appreciated from the following description of the embodiments, taken in conjunction with the accompanying drawings in which:

FIG. 1 shows an electroencephalogram measurement apparatus according to an embodiment of the present disclosure;

FIG. 2 shows an electroencephalogram measurement apparatus according to an embodiment of the present disclosure, on which a VR device and a band are put, as seen from above;

FIG. 3 shows an electroencephalogram measurement apparatus according to an embodiment of the present disclosure, on which a VR device and a band are put, as seen from the side;

FIG. 4 shows an electroencephalogram measurement apparatus according to an embodiment of the present disclosure, on which a VR device and a band are put, as seen from behind;

FIG. 5 shows a hook according to an embodiment of the present disclosure;

FIG. 6 shows a state in which connecting lines are connected to protruding sensors according to an embodiment of the present disclosure; and

FIG. 7 shows a state in which molds are disposed along the inner circumference surfaces of sensor holes according to an embodiment of the present disclosure.

### DETAILED DESCRIPTION

The present disclosure relates to an electroencephalogram measurement system using a Virtual Reality (VR) device and a band, and more particularly, to an electroencephalogram measurement system using a VR device and a band, which is capable of preventing interference between the band coupled to the VR device and an electroencephalogram measurement apparatus through sensor holes formed in the band in order to use the band in the electroencephalogram measurement apparatus. Hereinafter, embodiments of the present disclosure will be described in detail with reference to the accompanying drawings.

An electroencephalogram measurement system using a VR device and a band, according to the present disclosure, may include an electroencephalogram measurement apparatus 110, a band 120, and a VR device 140.

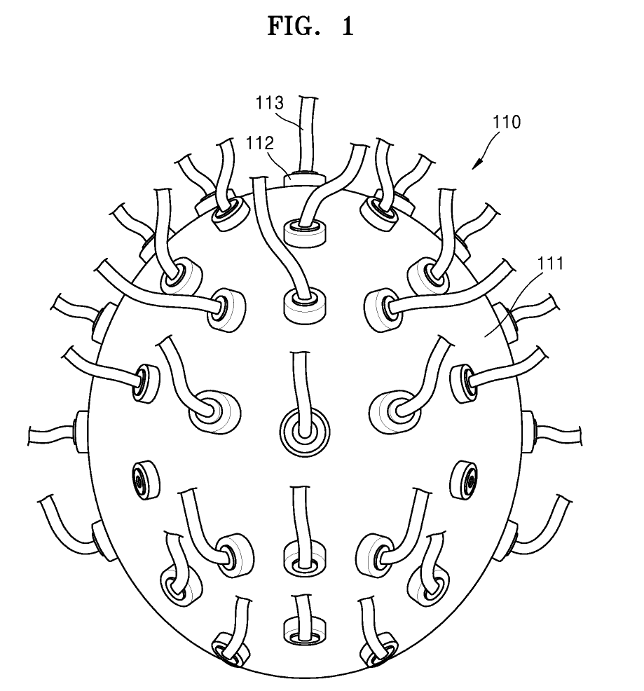

Referring to FIG. 1, the electroencephalogram measurement apparatus 110 may include a cover 111 that is put on a user's head, and a plurality of protruding sensors 112 protruding from the cover 111 at designated locations. The cover 111 may be used to rest the electroencephalogram measurement apparatus 110 on the user's head and configured to cover the user's entire head.

The protruding sensors 112 may protrude from the cover 111 at the designated locations to measure brain waves generated from the user's brain. The electroencephalogram measurement apparatus 110 may use various techniques for measuring and analyzing the brain, such as functional magnetic resonance imaging (fMRI), functional near infrared spectroscopy (fNIRS), electroencephalography (EEG), and eye-tracking. The user's brain waves may emerge from specific locations of the brain.

fNIRS, which is used to observe a blood flow rate of the brain, measures the blood flow rate of the brain using differences between oxygen-hemoglobin values of neuron actions. The brain is divided into a plurality of areas (the frontal lobe, the occipital lobe, etc.) according to their assigned functions, and the individual areas of the brain require large blood flow to activate the assigned functions. Blood flow is measured with optical fibers. That is, the user's brain waves emerge from the specific locations of the brain, and accordingly, the protruding sensors 112 for measuring brain waves need to be installed at the designated locations corresponding to the specific locations from which the user's brain waves emerge. That is, the installation locations of the protruding sensors 112 have been decided in advance.

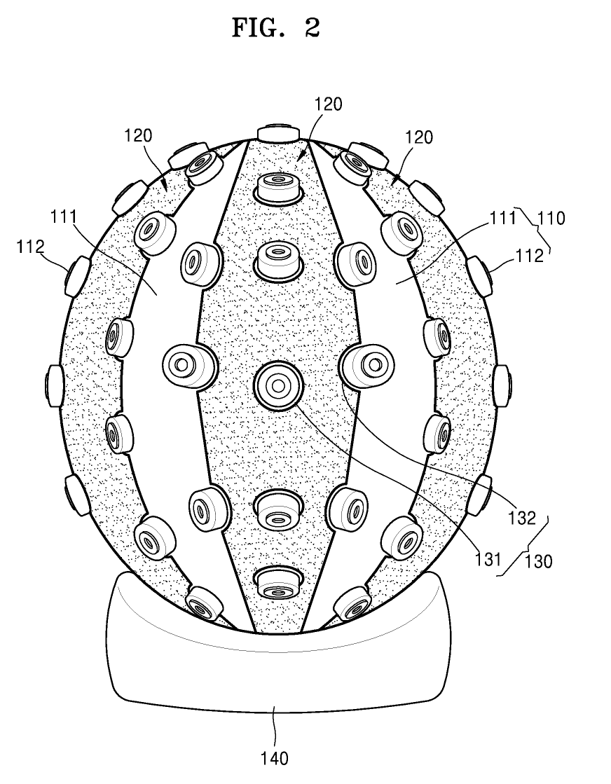

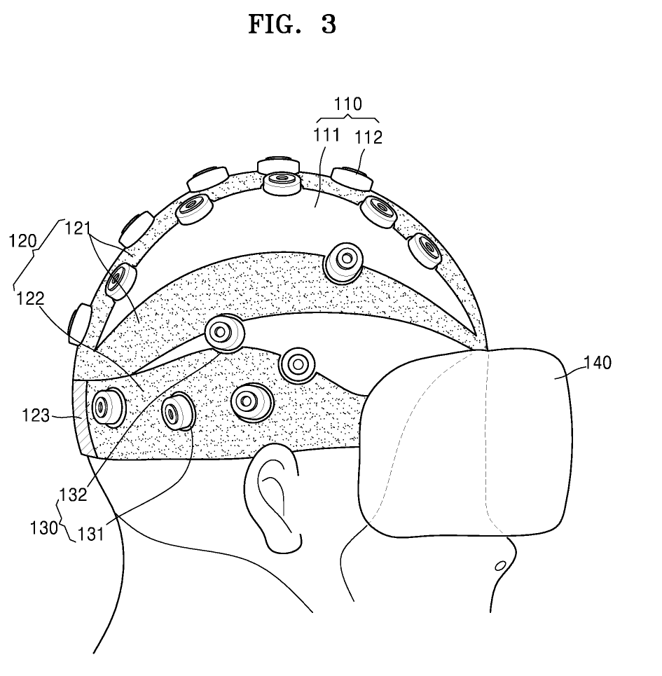

Referring to FIGS. 2 and 3, the band 120 may be put on the electroencephalogram measurement apparatus 110, and a plurality of sensor holes 130 may be arranged to correspond to the locations of the protruding sensors 112. The protruding sensors 112 may be installed to correspond to the specific locations from which brain waves emerge, as described above. The sensor holes 130 may be holes through which the protruding sensors 112 pass. In order for the protruding sensors 112 to pass through the sensor holes 130, the sensor holes 130 may need to be formed in correspondence to the locations of the protruding sensors 112.

The sensor holes 130 may include a first sensor hole 131 and a second sensor hole 132. The first sensor hole 131 may penetrate an inner portion of the band 120, and the second sensor hole 132 may penetrate an edge portion of the band 120. More specifically, the first sensor hole 131 may be in the shape of a circle since the first sensor hole 131 penetrates the inner portion of the band 120, and the second sensor hole 132 may be in the shape of a semicircle or an arc since the second sensor hole 132 penetrates the edge portion of the band 120.

The sensor holes 130 may be formed in the band 120 in correspondence to the locations of the protruding sensors 112, since the protruding sensors 112 installed in the designated locations pass through the sensor holes 130. Accordingly, the sensor holes 130 may be located in the inner portion of the band 120 and in the edge portion of the band 120.

Since the sensor holes 130 are formed, the protruding sensors 112 of the electroencephalogram measurement apparatus 110 may not interfere with the band 120 and the VR device 140. In a typical electroencephalogram measurement apparatus, sensors are pressed by a band so that pressure is generated in the sensors to cause measurement errors or inadequate data values. However, according to the present disclosure, since the protruding sensors 112 pass through the sensor holes 130, the protruding sensors 112 may be not pressed and thus making measurement errors or outputting inadequate data values is prevented.

The VR device 140 may be coupled to a front portion of the band 120 to be disposed around the user's eyes. The VR device 140 may be a VR device widely used in the related art, and accordingly, a detailed description thereof will be omitted.

The band 120 may be used to fix and support the VR device 140 having a heavy weight. In order to dispose the VR device 140 around the user's eyes and use the VR device 140, the VR device 140 may need to be fixed and supported. By putting the band 120 on the user's head, the VR device 140 may be fixed and supported, and accordingly, the VR device 140 may be stably used.

In order to easily put the band 120 on the user's head and to reinforce the tension of the band 120, the band 120 may be composed of an upward band 121 and a horizontal band 122. Since the sensor holes 130 are formed in the band 120, the tension of the band 120 may be reduced by the sensor holes 130.

In order to prevent the tension of the band 120 from being reduced, the upward band 122 may diverge into a plurality of pieces to extend toward the top of the user's head from the VR device 140, and the horizontal band 122 may diverge into two pieces to extend in a horizontal direction from the VR device 140. More specifically, the upward band 121 may diverge into three pieces, and the three pieces of the upward band 121 may reinforce the tension of the band 120, together with the horizontal band 122 extending in the horizontal direction.

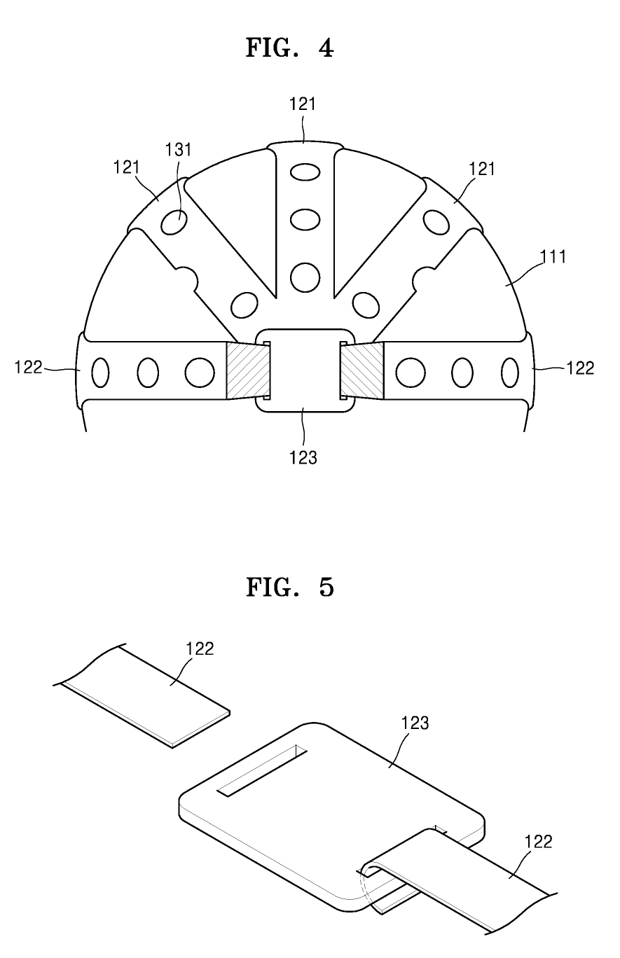

Hereinafter, directions in which the upward band 121 and the horizontal band 122 extend will be described with reference to FIGS. 3 and 4. The plurality of pieces of the upward band 121 may extend toward the top of the user's head from the VR device 140, and after the plurality of pieces of the upward band 121 pass the top of the user's head, the plurality of pieces of the upward band 121 may extend downward to converge on a hook 123. The two pieces of the horizontal band 122 may extend in the horizontal direction from the VR device 140, that is, along the side of the user's head and then converge on the hook 123.

The hook 123 may be at a location at which the upward band 121 and the horizontal band 122 converge. The upward band 121 may be fixed at the hook 123, whereas the horizontal band 122 may be detachably coupled to the hook 123. More specifically, in order for a user to easily put the band 120 on his/her head, the user may put the upward band 121 on his/her head earlier than the horizontal band 122. By first putting the upward band 121 fixed at the hook 123 on the user's head together with the hook 123, and then coupling the horizontal band 122 detachably coupled to the hook 123 to the hook 123, the band 120 may be worn on the user's head.

By coupling the horizontal band 122 to the hook 123 after putting the upward band 121 on the user's head, the user may easily wear the upward band 121. There may be various methods of coupling the horizontal band 122 to the hook 123. As shown in FIG. 5, holes may be formed in the hook 123, and the horizontal band 122 may be inserted into the holes to be coupled to the hook 123. However, a method of coupling/decoupling the horizontal band 122 to/from the hook 123 is not limited thereto. That is, various methods may be used which couple/decouple the horizontal band 122 to/from the hook 123.

A method of operating the electroencephalogram measurement system using the VR device and the band is as follows. The electroencephalogram measurement apparatus 110 may be first put on a user's head, and the protruding sensors 112 of the electroencephalogram measurement apparatus 110 may pass through the sensor holes 130 of the band 120 so that the band 120 and the VR device 140 may be worn on the user's head. Since the VR device 140 is coupled to the front portion of the band 120, the VR device 140 may be worn on the user's head together with the band 120.

When the band 120 and the VR device 140 are worn on the user's head, the protruding sensors 112 may pass through the sensor holes 130, and accordingly, the protruding sensors 112 may be prevented from interfering with the band 120 and the VR device 140.

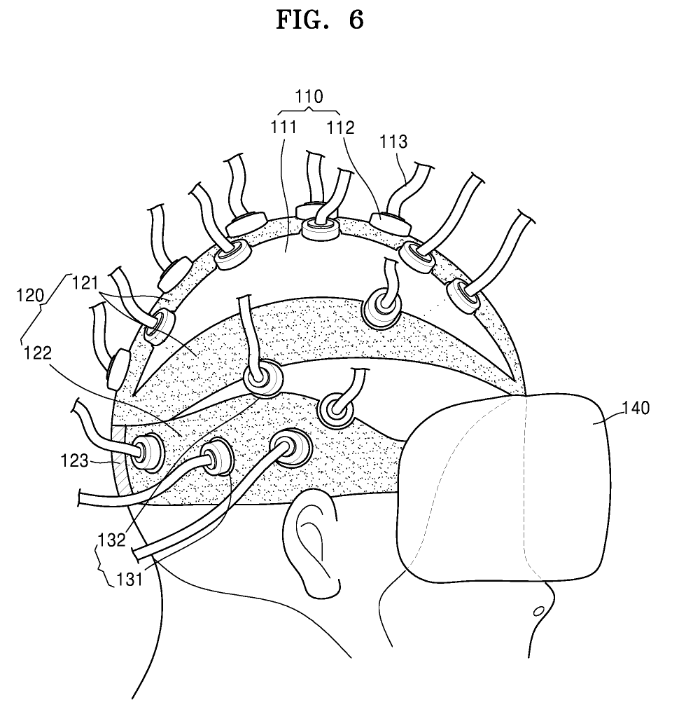

Referring to FIG. 6, a plurality of connecting lines 113 may be connected to the protruding sensors 112. The connecting lines 113 may be connected to the protruding sensors 112 to transmit signals generated by the protruding sensors 112 to an analysis apparatus. The connection lines 113 may be removably connected to the protruding sensors 112 after the band 120 and the VR device 120 are worn on the user's head.

There are difficulties in connecting connecting lines to sensors due to a fixing band of a VR device, and furthermore, the pressure of the fixing band due to the weight of the VR device has an adverse effect on the connecting lines. However, according to the present disclosure, the protruding sensors 112 may pass through the sensor holes 130 of the band 120 and be exposed to the outside, and accordingly, it may be possible to easily connect the connecting lines 113 to the protruding sensors 112 without any interference with the band 120.

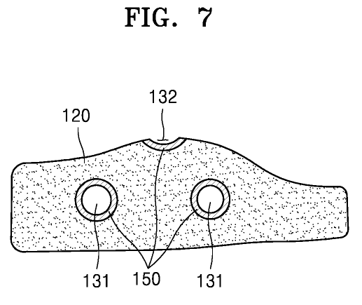

Referring to FIG. 7, a plurality of molds 150 may be coupled to the sensor holes 130 along the inner circumference surfaces of the sensor holes 130. Since each sensor hole 130 is in the shape of a circle or an arc, the sensor hole 130 may be damaged when tension is applied to the band 120. Particularly, the second sensor holes 132 formed in the shape of an arc may be seriously damaged.

The molds 150 may be coupled to the inner circumference surfaces of the sensor holes 130 in order to prevent the sensor holes 130 from being damaged. The molds 150 may be made of a rigid material and coupled to the inner circumference surfaces of the sensor holes 130 in correspondence of the shapes of the inner circumference surfaces of the sensor holes 130. Since the first sensor holes 131 are formed in the shape of a circle, the molds 150 coupled to the inner circumference surfaces of the first sensor holes 131 may also be formed in the shape of a circle. Also, since the second sensor holes 132 are formed in the shape of a semicircle or an arc, the molds 150 coupled to the inner circumference surfaces of the second sensor holes 132 may also be formed in the shape of a semicircle or an arc.

The electroencephalogram measurement system using the VR device and the band, according to the present disclosure, may have the following effects.

The sensor holes 130 may be formed in the band 120 in order to use the band 120 coupled to the VR device 140 in the electroencephalogram measurement apparatus 110. Therefore, the VR device 140 and the band 120 may be prevented from interfering with the electroencephalogram measurement apparatus 110. More specifically, the protruding sensors 112 of the electroencephalogram measurement apparatus 110 may operate without any interference with the band 120 for fixing the VR device 140.

Also, according to the present disclosure, by forming the sensor holes 130 in the band 120 in order to use the band 120 in the electroencephalogram measurement apparatus 110, it may be possible to connect the connecting lines 113 to the protruding sensors 112 without any interference with the VR device 140 and the band 120.

According to the present disclosure, the electroencephalogram measurement system using the VR device and the band may prevent interference between the band coupled to the VR device and the electroencephalogram measurement apparatus through the sensor holes formed in the band in order to use the band in the electroencephalogram measurement apparatus. Also, by forming the sensor holes in the band in order to use the band coupled to the VR device in the electroencephalogram measurement apparatus, it may be possible to connect the connecting lines to the protruding sensors without any interference with the VR device and the band.

It should be understood that embodiments described herein should be considered in a descriptive sense only and not for purposes of limitation. Descriptions of features or aspects within each embodiment should typically be considered as available for other similar features or aspects in other embodiments.

While one or more embodiments have been described with reference to the figures, it will be understood by those of ordinary skill in the art that various changes in form and details may be made therein without departing from the spirit and scope of the disclosure as defined by the following claims.

What is claimed is:

1. An electroencephalogram measurement system using a Virtual Reality (VR) device and a band for measuring brain waves using the VR device, the electroencephalogram measurement system using the VR device and the band comprising:
an electroencephalogram measurement apparatus comprising a cover that is worn on a user's head, and a protruding sensor protruding from the cover at a designated location;
a band that is put on the electroencephalogram measurement apparatus, the band having a sensor hole formed in correspondence to a location at which the protruding sensor protrudes; and
a VR device coupled to a front portion of the band, and disposed around the user's eyes,
wherein, after the electroencephalogram measurement apparatus is put on the user's head, the band and the VR device are worn on the user's head while the protruding sensor of the electroencephalogram measurement apparatus passes through the sensor hole of the band.

2. The electroencephalogram measurement system using the VR device and the band of claim 1, wherein the band comprises:
an upward band diverging into a plurality of pieces to extend toward the top of the user's head from the VR device; and
a horizontal band diverging into two pieces to extend in a horizontal direction from the VR device,
wherein the upper band is put on the user's head earlier than the horizontal band.

3. The electroencephalogram measurement system using the VR device and the band of claim 2, further comprising a hook on which the upward band and the horizontal band converge,
wherein the horizontal band is detachably coupled to the hook.

4. The electroencephalogram measurement system using the VR device and the band of claim 2, wherein the sensor hole comprises a first sensor hole penetrating an inner portion of the band, and a second sensor hole penetrating an edge portion of the band.

5. The electroencephalogram measurement system using the VR device and the band of claim 1, wherein a mold is coupled to the sensor hole along an inner circumference surface of the sensor hole.

6. The electroencephalogram measurement system using the VR device and the band of claim 1, further comprising a connecting line connected to the protruding sensor, and configured to transmit a signal generated by the protruding sensor,
wherein the connection line is connected to the protruding sensor after the band and the VR device are worn on the user's head.
